(19) United States
(12) Patent Application Publication
(10) Pub. No.: US 2021/0212660 A1
(43) Pub. Date: Jul. 15, 2021

(54) ULTRASOUND DIAGNOSIS APPARATUS AND PROGRAM

(71) Applicant: Hitachi, Ltd., Tokyo (JP)

(72) Inventor: Tomofumi Nishiura, Tokyo (JP)

(73) Assignee: Hitachi, Ltd., Tokyo (JP)

(21) Appl. No.: 16/898,635

(22) Filed: Jun. 11, 2020

(30) Foreign Application Priority Data
Jan. 9, 2020 (JP) 2020-001794

(51) Int. Cl. A61B 8/00; A61B 8/08; A61B 8/15

(52) U.S. Cl. CPC A61B 8/4254; A61B 8/485; A61B 8/15; A61B 8/5207; A61B 8/461

(57) ABSTRACT

An ultrasound diagnosis apparatus includes a processor for performing grouping processing and representative frame selection processing on a plurality of ultrasound frames that are sequentially generated with the passage of time. The grouping processing is processing that identifies a group of interest that is composed of a plurality of frames that satisfy at least one predetermined condition of interest. The representative frame selection processing is processing that selects a representative frame from among the plurality of frames that constitute the group of interest. The at least one predetermined condition of interest includes a condition that each of the plurality of frames includes data indicating a specific region identified by region recognition processing in which a characteristic region in an image is recognized as the specific region. The representative frame selection processing includes processing that selects the representative frame based on geometrical properties of the specific regions.

### CROSS REFERENCE TO RELATED APPLICATION

This application claims priority to Japanese Patent Application No. 2020-001794 filed on Jan. 9, 2020, which is incorporated herein by reference in its entirety including the specification, claims, drawings, and abstract.

### TECHNICAL FIELD

The present disclosure relates to an ultrasound diagnosis apparatus and program and, in particular, to processing for identifying, from among a plurality of frames representing ultrasound images, a group of frames that satisfy one or more predetermined conditions.

### BACKGROUND

Ultrasound diagnosis apparatuses are widely used as apparatuses for observing a subject. An ultrasound diagnosis apparatus sequentially generates frame data (hereinafter referred to as frames) representing ultrasound images of a subject by transmission and reception of ultrasound with the passage of time, and displays on a monitor images based on the frames with the passage of time.

Some ultrasound diagnosis apparatuses include a cine memory for storing frames that are sequentially generated with the passage of time. Frames are sequentially generated with the passage of time, images based on the frames are sequentially displayed on the monitor with the passage of time, and frames corresponding to the displayed images are stored in the cine memory. The cine memory stores, along with the latest frame, a series of frames that are generated during a certain previous period of time. The ultrasound diagnosis apparatus designates one of the frames stored in the cine memory based on an operation of a user, and displays on the monitor an image based on the designated frame.

Japanese patent publications, including JP 2016-97256 A, JP 2016-112033 A, JP 2018-339 A, and JP 2019-24925 A, disclose techniques for evaluating tissue of a subject based on frames that are sequentially generated by transmission and reception of ultrasound.

### SUMMARY

In the processing of designating one of the frames stored in the cine memory based on an operation of a user followed by displaying an image based on the designated frame on the monitor, a frame that is to be displayed is designated from among a plurality of frames stored in the cine memory. Examples of the frame that is to be displayed include frames including data indicating regions that should be of interest in a subject, such as regions where a sign of cancer, hepatic cirrhosis, or other diseases appears. When a large number of frames are stored in the cine memory in a random manner, the designating and displaying operation may impose a significant burden on the user.

The present disclosure is directed toward designating, from among a plurality of frames, a frame corresponding to a region that should be of interest in a subject, through a simple process.

According to one aspect of the present disclosure, there is provided an ultrasound diagnosis apparatus including a processor for performing, on a plurality of ultrasound frames that are sequentially generated by transmission and reception of ultrasound, grouping processing of identifying a group of interest that is composed of a plurality of frames that satisfy at least one predetermined condition of interest; and representative frame selection processing of selecting a representative frame from among the plurality of frames that constitute the group of interest. The at least one predetermined condition of interest includes a condition that each of the plurality of frames includes data indicating a specific region identified by region recognition processing in which a characteristic region in an image is recognized as the specific region. The representative frame selection processing includes processing that selects the representative frame based on geometrical properties of the specific regions.

By employing the present disclosure, a frame corresponding to a region that should be of interest in a subject can be designated from among a plurality of frames through a simple process.

### BRIEF DESCRIPTION OF DRAWINGS

Embodiments of the present disclosure will be described based on the following figures, wherein:

FIG. 1 illustrates a structure of an ultrasound diagnosis apparatus;

FIG. 2 illustrates a structure of an ultrasound image generation module, together with an ultrasound transceiver, a lesion candidate detection module, and a display;

FIG. 3 conceptually illustrates ultrasound images and frames;

FIG. 4 illustrates a structure of the lesion candidate detection module, together with a cine memory and a control module;

FIG. 5 illustrates an example of a detection information table;

FIG. 6 illustrates an example of a group-of-interest table;

FIG. 7 illustrates an example of a representative frame table;

FIG. 8 illustrates an example of a correspondence relationship between the detection information table and the representative frame table;

FIG. 9 illustrates a plurality of frames schematically representing image data;

FIG. 10 illustrates a structure of an ultrasound diagnosis apparatus; and

FIG. 11 illustrates lesion candidate regions for individual frames.

### DESCRIPTION OF EMBODIMENTS

### (1) Structure and Basic Operation of Ultrasound Diagnosis Apparatus

An ultrasound diagnosis apparatus according to an embodiment of the present disclosure will be described with reference to the accompanying drawings. The same features illustrated in two or more drawings are denoted by the same reference numerals, and their descriptions are not repeated.

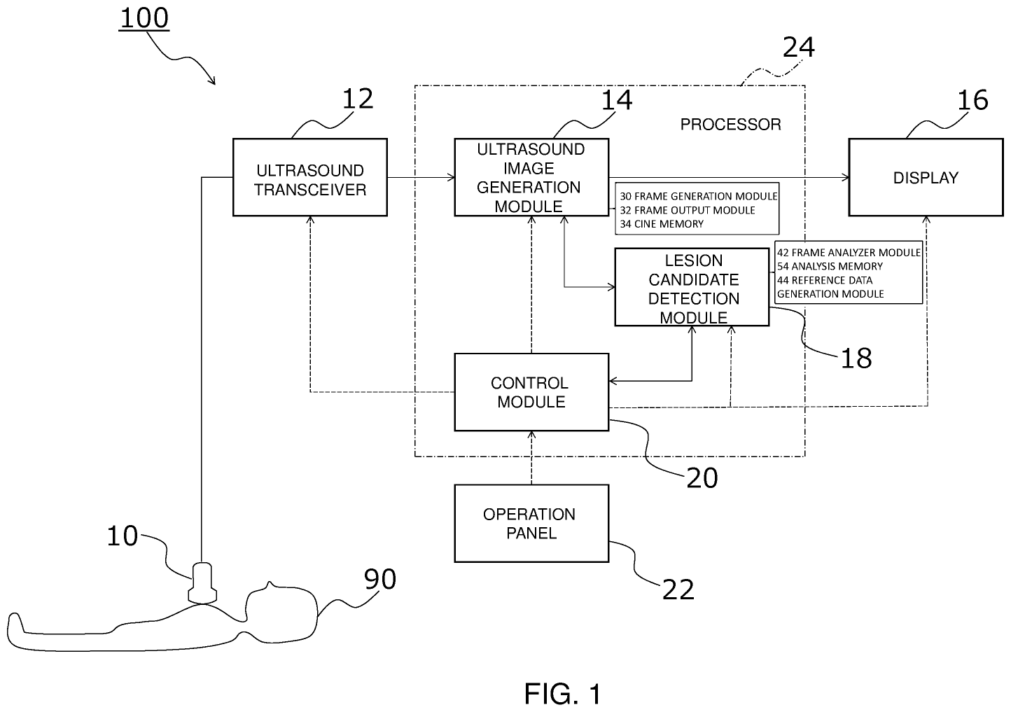

FIG. 1 illustrates a structure of an ultrasound diagnosis apparatus 100 according to an embodiment of the present disclosure. The ultrasound diagnosis apparatus 100 includes an ultrasound probe 10, an ultrasound transceiver 12, a processor 24, a display 16, and an operation panel 22. The processor 24 includes a control module 20, an ultrasound image generation module 14, and a lesion candidate detection module 18. The processor 24 executes either an ultrasound diagnosis program that is externally downloaded and stored therein, or an ultrasound diagnosis program that is prestored therein, to thereby implement the control module 20, the ultrasound image generation module 14, and the lesion candidate detection module 18. Examples of the display 16 serving as a monitor may include a liquid crystal display and an organic EL display.

The operation panel 22 may include, for example, a keyboard, a mouse, a touch panel, a lever, and a rotary knob. The operation panel 22 outputs operation information to the control module 20 in response to an operation of a user. Based on the operation information, the control module 20 controls the ultrasound transceiver 12, the ultrasound image generation module 14, the lesion candidate detection module 18, and the display 16. The operation panel 22 may be a touch panel display that is integral with the display 16.

In response to control from the control module 20, the ultrasound transceiver 12, the ultrasound image generation module 14, the lesion candidate detection module 18, and the display 16 operate as will be described below. The ultrasound probe 10 includes a plurality of transducers, and the ultrasound transceiver 12 outputs transmission signals in the form of electrical signals to individual ones of the plurality of transducers. The plurality of transducers transmit ultrasound to a subject 90 in accordance with the individually supplied transmission signals. The plurality of transducers receive ultrasound reflected by the subject 90 and output reception signals in the form of electrical signals to the ultrasound transceiver 12.

The ultrasound transceiver 12 adjusts the delay time of the transmission signals that are output to individual ones of the plurality of transducers, thereby directing ultrasound transmitted from the plurality of transducers to the subject 90 toward a specific direction to form an ultrasound beam. The ultrasound transceiver 12 subjects the reception signals output from the plurality of transducers to phasing addition to thereby cause the plurality of reception signals based on the ultrasound received from the direction of the ultrasound beam to strengthen each other. The ultrasound transceiver 12 outputs phased and added reception signals that are obtained through phasing addition to the ultrasound image generation module 14.

The ultrasound transceiver 12 varies the delay time of the transmission signals that are output to individual ones of the plurality of transducers, thereby scanning an ultrasound beam that is formed in the subject 90. As the ultrasound beam is scanned, the reception signals that are output from the plurality of transducers are subjected to phasing addition to output, to the ultrasound image generation module 14, reception signals that are phased and added with respect to the directions or the positions of the ultrasound beam.

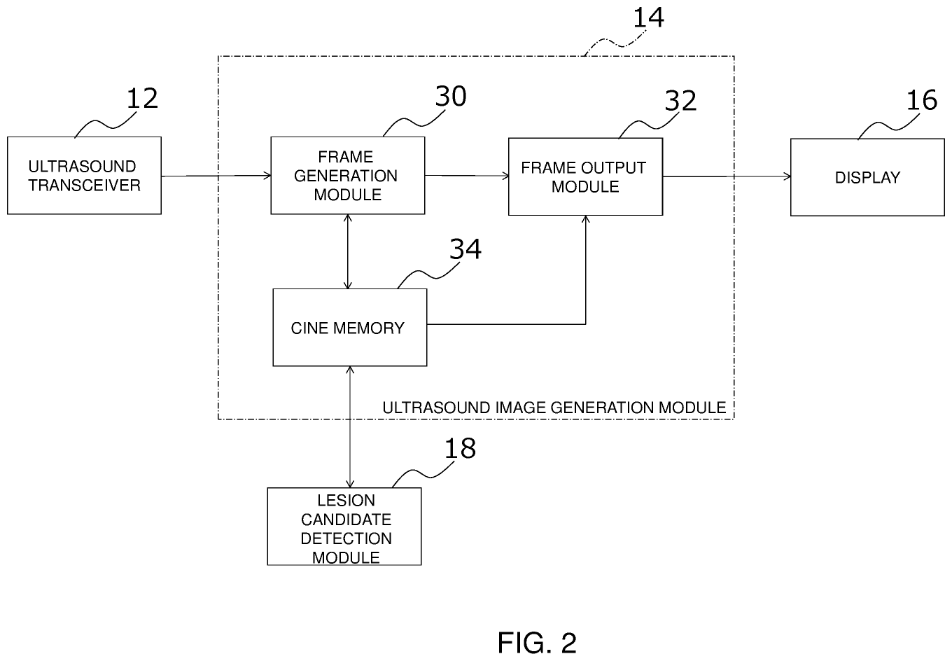

FIG. 2 illustrates a structure of the ultrasound image generation module 14, together with the ultrasound transceiver 12, the lesion candidate detection module 18, and the display 16. The ultrasound image generation module 14 includes a frame generation module 30, a frame output module 32, and a cine memory 34. The frame generation module 30 generates frames (ultrasound frames) representing ultrasound images based on the reception signals that are phased and added with respect to the directions or the positions of the ultrasound beam. The frame generation module 30 may generate one frame each time an ultrasound beam is scanned with respect to a tomographic plane of the subject 90. One frame represents one ultrasound image.

The frame generation module 30 outputs frames to the frame output module 32 and the cine memory 34 sequentially with the passage of time at a predetermined frame rate. In this embodiment, the frame rate is defined as the number of frames that are output from the frame generation module 30 per unit time. The cine memory 34 stores, in addition to the latest frame, the previous N−1 frames. When the cine memory 34 stores N frames, to store a newly generated frame in the cine memory 34, the oldest frame is deleted, and the latest frame is stored in the cine memory 34.

The operation modes of the ultrasound diagnosis apparatus 100 will be described with reference to FIGS. 1 and 2. The operation modes of the ultrasound diagnosis apparatus 100 include a real-time measurement mode and a freeze mode. In the real-time measurement mode, as an ultrasound beam is repeatedly scanned with respect to the tomographic plane of the subject 90, the frame generation module 30 sequentially generates frames, and the display 16 sequentially displays ultrasound images based on the sequentially generated frames. In the freeze mode, an ultrasound image based on the last generated frame or a frame that is read from the cine memory 34 is kept displayed on the display 16. In the freeze mode, the operation in which the ultrasound transceiver 12 outputs transmission signals to the ultrasound probe 10 and in which the ultrasound transceiver 12 obtains reception signals from the ultrasound probe 10 is stopped, and frames are kept stored in the cine memory 34.

During operation in the real-time measurement mode, the frame output module 32 outputs frames that are output from the frame generation module 30 sequentially with the passage of time, to the display 16 sequentially with the passage of time. The display 16 displays ultrasound images based on frames that are sequentially output from the frame output module 32.

During operation in the freeze mode, in accordance with the user's operation on the operation panel 22, the control module 20 designates one of the frames that are stored in the cine memory 34 and causes the frame output module 32 to read the designated frame. The frame output module 32 reads the frame that is designated by the control module 20 from the cine memory 34 and outputs it to the display 16. The display 16 displays an ultrasound image based on the frame that is output from the frame output module 32.

Example procedures for performing a diagnosis of the subject 90 through the operation in the real-time measurement mode and through the operation in the freeze mode will be described below. When the ultrasound diagnosis apparatus 100 operates in the real-time measurement mode, the user moves the ultrasound probe 10 on the surface of the subject 90 while keeping the ultrasound probe 10 in contact with the subject 90. In other words, the user causes the ultrasound probe 10 to perform scanning on the subject 90 by the user's hand's motion.

As described above, while manual scanning is being performed by the ultrasound probe 10, frames are generated sequentially with the passage of time, and ultrasound images based on the frames are displayed on the display 16. The display 16 displays a moving image in which ultrasound images vary according to a predetermined frame rate. The frames generated by the frame generation module 30 are stored in the cine memory 34.

When the ultrasound diagnosis apparatus 100 operates in the real-time measurement mode, the operation mode of the ultrasound diagnosis apparatus 100 may be switched to the freeze mode in accordance with the user's operation on the operation panel 22. For example, upon identification of a lesion candidate region (specific region) where a disease such as cancer or hepatic cirrhosis is suspected in ultrasound images displayed on the display 16 through the operation in the real-time measurement mode, the user operates the operation panel 22 to switch the operation mode of the ultrasound diagnosis apparatus 100 from the real-time measurement mode to the freeze mode. As a result, the ultrasound diagnosis apparatus 100 changes to a state in which an ultrasound image based on the last generated frame is displayed on the display 16. In this state, as will be described below, an ultrasound image based on a frame that is read from the cine memory 34 in response to the user's designation can be displayed on the display 16.

As described above, the processor 24 performs display processing of storing a plurality of frames in the cine memory 34 and sequentially displaying ultrasound images based on the plurality of frames on the display 16 with the passage of time. The processor 24 further performs freeze processing of stopping the display processing based on an operation of a user and keeping a state of displaying on the display 16 either an ultrasound image that was displayed on the display 16 when the operation of the user has occurred or an ultrasound image based on a previously generated frame.

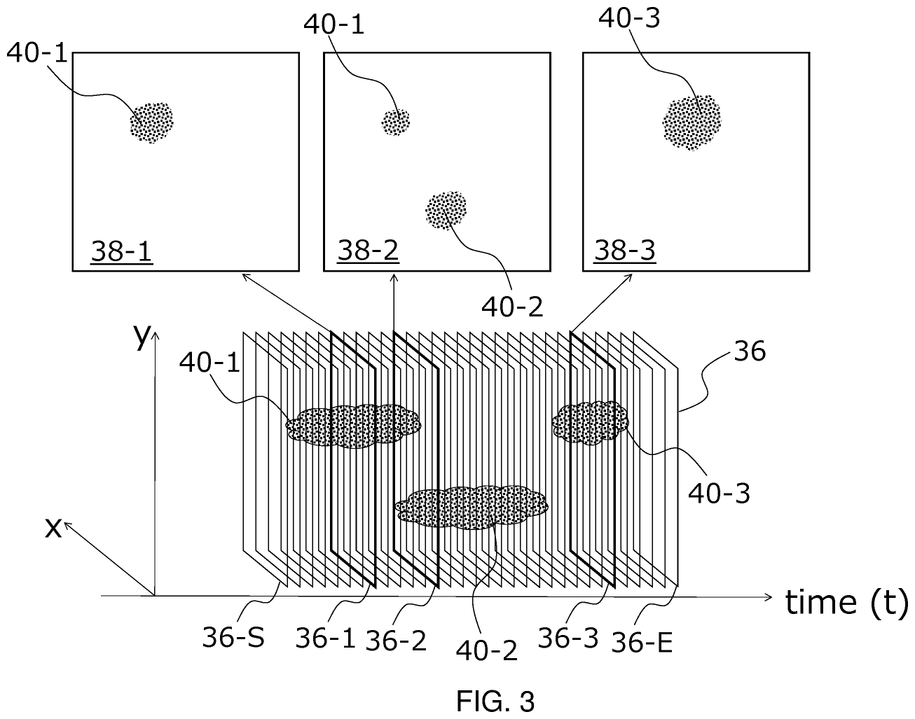

The upper half of FIG. 3 conceptually illustrates ultrasound images that are displayed on the display 16 in the freeze mode. The lower half of FIG. 3 conceptually illustrates frames 36 that are stored in the cine memory 34 in the form of two-dimensional ultrasound images. The frames 36 are frames obtained during operation in the real-time measurement mode when the ultrasound probe 10 is linearly moved on the subject 90 at a constant speed. The horizontal axis is a temporal axis (axis t), and an x-y plane is defined as vertical to the temporal axis. An ultrasound beam is scanned in a plane that is parallel with the x-y plane, ultrasound images represented by the frames 36 extend in parallel with the x-y plane, and the frames 36 are successive along the temporal axis. A frame 36-S that is located leftmost is a frame that is first stored in the cine memory 34, and a frame 36-E that is located rightmost is a frame that is last stored in the cine memory 34.

When, in response to an operation of the operation panel 22 illustrated in FIG. 1, the operation of the ultrasound diagnosis apparatus 100 is set to the freeze mode, the display 16 displays an ultrasound image based on the frame 36-E that is last stored in the cine memory 34. If a frame 36-1 is designated in response to an operation of the operation panel 22, the display 16 displays an ultrasound image 38-1. Similarly, if a frame 36-2 or 36-3 is designated, the display 16 displays an ultrasound image 38-2 or 38-3.

The lower half of FIG. 3 illustrates lesion candidate regions 40-1 to 40-3 for individual frames. Each of the lesion candidate regions is a specific region for which pixels of a frame have pixel values different from average pixel values of surrounding pixels, and is defined through region recognition processing of recognizing a characteristic region in an image. Examples of the region recognition processing include binarization processing, pattern matching, and region division, which will be described below.

The lesion candidate region 40-1 appears in the ultrasound image 38-1. The lesion candidate regions 40-1 and 40-2 appear in the ultrasound image 38-2. The lesion candidate region 40-3 appears in the ultrasound image 38-3. One of the frames stored in the cine memory 34 is designated by the user, and an ultrasound image is displayed in accordance with the designated frame to thereby diagnose one or more lesion candidate regions.

### (2) Operation of Lesion Candidate Detection Module in Real-Time Measurement Mode

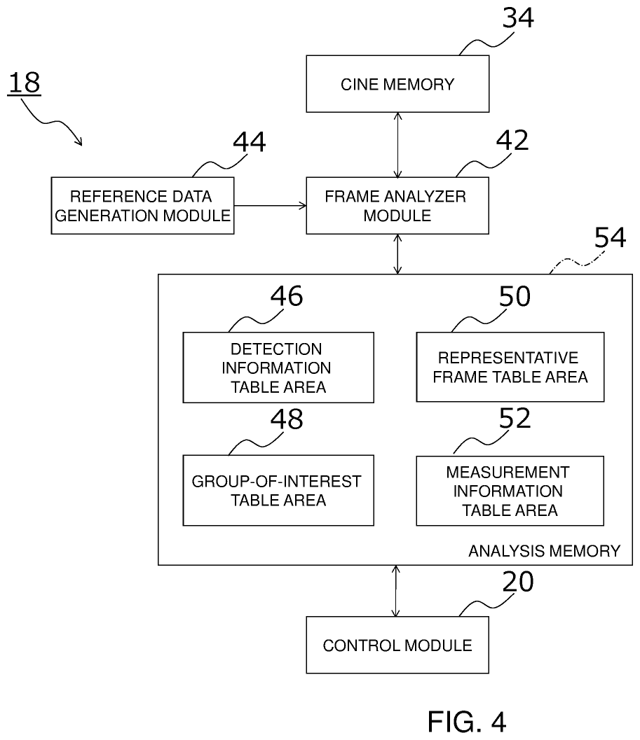

FIG. 4 illustrates a structure of the lesion candidate detection module 18, together with the cine memory 34 and the control module 20. The lesion candidate detection module 18 includes a frame analyzer module 42, an analysis memory 54, and a reference data generation module 44. The following description will describe the operation of the lesion candidate detection module 18 in the ultrasound diagnosis apparatus 100 that operates in the real-time measurement mode.

When one frame is newly stored in the cine memory 34, the frame analyzer module 42 generates detection information for that frame. The detection information includes a frame identification number serving as information for identifying the frame (frame identification information), the position of the lesion candidate region, and the size of the lesion candidate region, in association with each other. The position of the lesion candidate region is defined as, for example, the position of the center of gravity of the lesion candidate region. The size of the lesion candidate region is defined by, for example, the area of the lesion candidate region, the length of the maximum diameter, and the length of the minimum diameter. The diameter of the lesion candidate region is defined as, for example, a distance between two parallel straight lines having the lesion candidate region between them.

The frame analyzer module 42 may identify a lesion candidate region based on an ultrasound image represented by a frame through binarization processing as will be described below. The frame analyzer module 42 may perform binarization processing that sets pixel values for a region where the pixel values are greater than a predetermined binarization threshold value to 1 and sets pixel values that are less than or equal to the binarization threshold value to 0 to thereby identify the region for which the binarization processing has set the pixel values to 0 as a lesion candidate region.

The frame analyzer module 42 may identify a lesion candidate region through pattern matching as will be described below. The reference data generation module 44 stores or generates reference data that represent a plurality of different patterns of lesion candidate regions that differ in pixel values, sizes, shapes, or other parameter. The frame analyzer module 42 obtains the reference data from the reference data generation module 44 and determines a degree of approximation between each of the plurality of different patterns of lesion candidate regions and an ultrasound image represented by a frame. The degree of approximation may be a correlation value that is determined through correlation calculation of an image representing a pattern of a lesion candidate region and an ultrasound image represented by a frame. The frame analyzer module 42 identifies a lesion candidate region in an ultrasound image based on a pattern having a correlation value that is greater than a predetermined value.

The frame analyzer module 42 may identify a lesion candidate region through region division as will be described below. The region division is processing whereby a region having a predetermined characteristic regarding shape, size, pixel value, or other parameters is extracted from an ultrasound image. The region division is performed using reference data, which are either generated or stored by the reference data generation module 44. The frame analyzer module 42 obtains the reference data from the reference data generation module 44 and identifies a lesion candidate region by performing the region division on an ultrasound image represented by a frame.

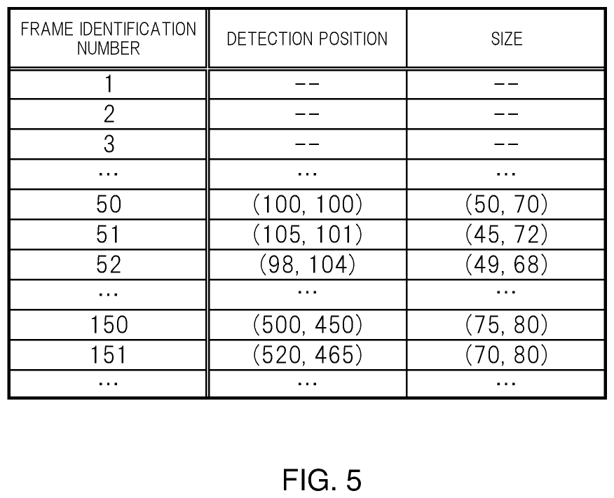

The frame analyzer module 42 generates detection information for each of the frames that are sequentially stored in the cine memory 34. The frame analyzer module 42 further generates a detection information table listing geometrical properties such as the position and the size of each lesion candidate region that are associated with a frame identification number of a corresponding frame, and stores the generated detection information table in a detection information table area 46 of the analysis memory 54. FIG. 5 illustrates an example of the detection information table. Each of the frames is assigned a frame identification number in accordance with the order in which it is stored in the cine memory 34. In the illustrated example, symbol “--” indicates that, as frames assigned frame identification numbers 1, 2, and 3 have no detected lesion candidate region, neither the position of a lesion candidate region nor the size of a lesion candidate region is determined.

Frames assigned frame identification numbers 50, 51, 52, 150, and 151 have associated therewith detection position and size that are determined by the frame analyzer module 42. The detection position is expressed as an x-y coordinate value in the form of “(x, y)”, where x represents an x axis coordinate value and y represents a y axis coordinate value. The size is expressed in the form of “(Ra, Rb)”, where Ra represents the minimum diameter, and Rb represents the maximum diameter. The size of a lesion candidate region may be expressed as the area of the lesion candidate region.

### (3) Operation of Lesion Candidate Detection Module in Freeze Mode

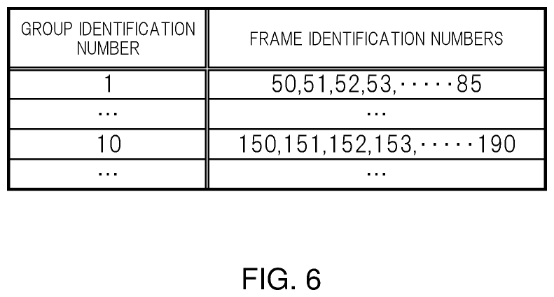

Next, processing performed by the frame analyzer module 42 in the ultrasound diagnosis apparatus 100 that operates in the freeze mode will be described below mainly with reference to FIG. 4, and where appropriate with reference to FIGS. 6 to 8. The cine memory 34 stores a plurality of previous frames obtained during a period of time going back from the time when the operation mode of the ultrasound diagnosis apparatus 100 is set to the freeze mode. The frame analyzer module 42 refers to the detection information table and performs grouping processing on frame groups that are composed of a plurality of frames stored in the cine memory 34.

The grouping processing is processing that identifies, from among a plurality of frames that constitute frame groups, a group of interest that is composed of frames that satisfy one or more predetermined conditions of interest. The conditions of interest may include the condition that each of the frames includes data indicating a detected lesion candidate region. The conditions for interest may include the condition that each of the frames includes data indicating a detected lesion candidate region, wherein lesion candidate regions in frames having adjacent frame identification numbers are located close to each other. The following description will describe an example in which the latter condition of interest is employed.

In this embodiment, the state in which lesion candidate regions are located close to each other may be defined as a state in which a distance between the position of a lesion candidate region indicated by data included in one of two frames having adjacent frame identification numbers (hereinafter referred to as adjacent frames) and the position of a lesion candidate region indicated by data included in the other frame is less than or equal to a predetermined threshold value. The state in which lesion candidate regions are located close to each other may also be defined as a state in which the overlapping ratio indicating the extent of overlap between a lesion candidate region indicated by data included in one of adjacent frames and a lesion candidate region indicated by data included in the other frame is greater than a predetermined threshold value. In this embodiment, the overlapping ratio is defined as a ratio of the area where a projection image of a lesion candidate region indicated by data included in one of adjacent frames on the x-y plane and a projection image of a lesion candidate region indicated by data included in the other frame on the x-y plane overlap each other to the total area of the areas of the lesion candidate regions indicated by data included in the adjacent frames. The state in which lesion candidate regions are located close to each other may also be defined as a state in which a distance between the position of a lesion candidate region indicated by data included in one of adjacent frames and the position of a lesion candidate region indicated by data included in the other frame is less than or equal to a predetermined threshold value, and in which the ratio of overlap between lesion candidate regions indicated by data included in adjacent frames is greater than a predetermined threshold value.

As described above, the grouping processing includes processing that identifies, from among a plurality of frames, frames that constitute a group of interest based on the positional relationship of lesion candidate regions (specific regions) for frames that are adjacent to each other on the temporal axis.

The frame analyzer module 42 generates a group-of-interest table listing frame groups that are identified as groups of interest, and stores the generated group-of-interest table in a group-of-interest table area 48 of the analysis memory 54. The group-of-interest table lists group identification numbers each identifying a group of interest and associated with frame identification numbers of a plurality of frames that constitute the group of interest. FIG. 6 illustrates an example of the group-of-interest table. In the illustrated example, frame identification numbers 50, 51, 52, 53, . . . 85 are associated with a group identification number 1. Frame identification numbers 150, 151, 152, 153, . . . 190 are associated with a group identification number 10. In other words, a group of frames that are identified by the frame identification numbers 50, 51, 52, 53, . . . 85 constitute a group of interest that is identified by the group identification number 1. A group of frames that are identified by the frame identification numbers 150, 151, 152, 153, . . . 190 constitute a group of interest that is identified by the group identification number 10.

The frame analyzer module 42 performs representative frame selection processing on a plurality of frames that constitute a group of interest. The representative frame selection processing includes processing that selects a representative frame from among a plurality of frames that constitute a group of interest based on geometrical properties of lesion candidate regions. In other words, the frame analyzer module 42 refers to the group-of-interest table stored in the group-of-interest table area 48 and the detection information table stored in the detection information table area 46, and selects a representative frame from among a plurality of frames that constitute a group of interest based on geometrical properties of lesion candidate regions.

For example, the frame analyzer module 42 may select, as the representative frame, a frame that is located at the midpoint on the temporal axis in a time period during which a plurality of frames that constitute a group of interest are generated. In other words, assuming that a group of interest is composed of M+1 frames having frame identification numbers K to K+M, if M is an even number, the frame analyzer module 42 may select a frame having a frame identification number K+M/2 as the representative frame. If M is an odd number, the frame analyzer module 42 may select a frame having a frame identification number K+(M−1)/2 or K+(M+1)/2 as the representative frame. M is an integer of 2 or greater.

From among a plurality of frames that constitute a group of interest, the frame analyzer module 42 may select a frame including a lesion candidate region having a largest maximum diameter as the representative frame, and may select a frame including a lesion candidate region having a largest area as the representative frame. From among a plurality of frames that constitute a group of interest, the frame analyzer module 42 may select as the representative frame one of two adjacent frames having a minimum absolute value of the difference between the areas of lesion candidate regions in adjacent frames.

The frame analyzer module 42 may select a frame that includes the center of gravity of a lesion candidate region in a three-dimensional space as the representative frame. A lesion candidate region in a three-dimensional space is a three-dimensional lesion candidate region indicated by data included in a frame group in a three-dimensional xyt space defined by the temporal axis t, the x axis, and the y axis.

The frame analyzer module 42 generates a representative frame table listing representative frame identification numbers each identifying a representative frame and associated with a group identification number, and stores the generated representative frame table in a representative frame table area 50 of the analysis memory 54.

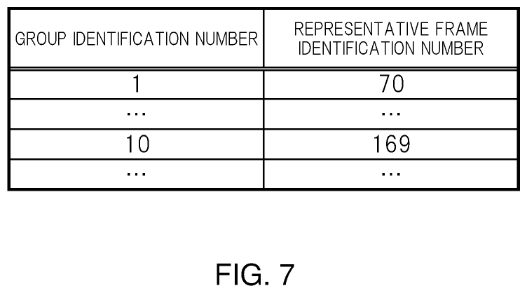

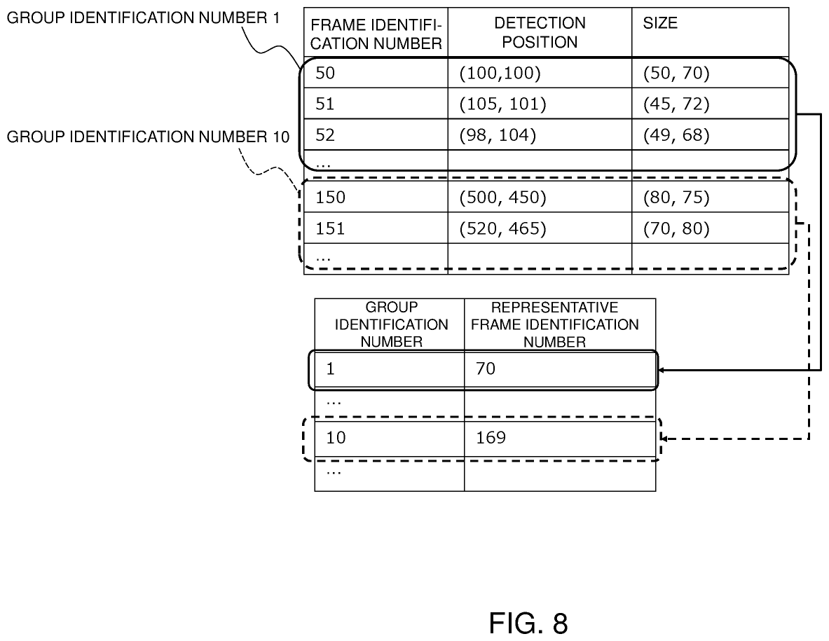

FIG. 7 illustrates an example of the representative frame table. FIG. 8 illustrates an example of a correspondence relationship between the detection information table and the representative frame table. As illustrated in FIG. 7, a representative frame identification number 70 is associated with the group identification number 1, and a representative frame identification number 169 is associated with the group identification number 10. In other words, the representative frame of a group of interest identified by the group identification number 1 is a frame identified by the representative frame identification number 70. The representative frame of a group of interest identified by the group identification number 10 is a frame identified by the representative frame identification number 169.

FIG. 8 indicates that, as the representative frame for the group of interest that is composed of the frames identified by the frame identification numbers 50, 51, 52, . . . 85, the frame identified by the representative frame identification number 70 has been selected. FIG. 8 further indicates that, as the representative frame for the group of interest that is composed of the frames identified by the frame identification numbers 150, 151, 152, . . . 190, the frame identified by the representative frame identification number 169 has been selected.

The frame analyzer module 42 may perform lesion measurement processing on a lesion candidate region indicated by data included in a representative frame. In other words, in addition to selecting a representative frame, the frame analyzer module 42 may determine the area of the lesion candidate region indicated by data included in the representative frame, the length of the perimeter, the maximum diameter, the minimum diameter, the average, maximum, and minimum values of pixel values in the lesion candidate region, or other lesion measurement information. The detection information that has been previously determined may be used as some of the lesion measurement information. The frame analyzer module 42 generates a measurement information table listing lesion measurement information associated with representative frame identification numbers, and stores the generated measurement information table in a measurement information table area 52 of the analysis memory 54.

### (4) Processing of Displaying Ultrasound Image Based on Representative Frame

Processing of displaying an ultrasound image based on a representative frame on the display 16 in the ultrasound diagnosis apparatus 100 that operates in the freeze mode will be described below with reference to FIGS. 1, 2, 4, and 9. The control module 20 refers to the representative frame table area 50 of the analysis memory 54, and outputs representative frame information for enabling the user to designate a representative frame to the display 16. The display 16 displays an image in accordance with the representative frame information.

The representative frame information may be information representing a list including a sequence of representative frame identification numbers. Upon an operation by the user of designating a representative frame identification number on the operation panel 22, the control module 20 controls the ultrasound image generation module 14, and causes the display 16 to display an ultrasound image represented by a representative frame corresponding to the representative frame identification number. In other words, the frame output module 32 of the ultrasound image generation module 14 illustrated in FIG. 2 reads the representative frame from the cine memory 34 and causes the display 16 to display the ultrasound image.

In addition to displaying an ultrasound image represented by a representative frame on the display 16, the control module 20 may refer to the measurement information table to obtain lesion measurement information based on the representative frame identification number to cause the display 16 to display the lesion measurement information for the representative frame.

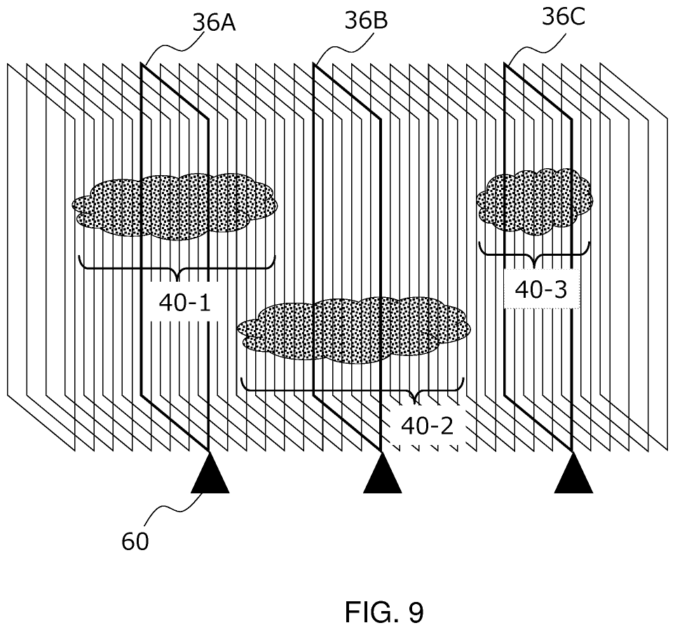

The representative frame information may be image data for schematically displaying, as illustrated in FIG. 9, a plurality of frames stored in the cine memory 34. In the image illustrated in FIG. 9, a frame 36A is the representative frame corresponding to the lesion candidate region 40-1. A frame 36B is the representative frame corresponding to the lesion candidate region 40-2, and a frame 36C is the representative frame corresponding to the lesion candidate region 40-3. The frames 36A, 36B, and 36C serving as the representative frames are depicted by thicker lines than other frames. Buttons 60 for designating one of the frames 36A, 36B, and 36C are displayed below the frames 36A, 36B, and 36C serving as the representative frames.

The operation by the user of designating a representative frame on the operation panel 22 may be performed by, for example, moving a cursor and clicking one of the buttons 60 located below the frames 36A, 36B, and 36C serving as the representative frames on the image displayed by the display 16. One of the frames 36A, 36B, and 36C may be designated by an operation of a keyboard included in the operation panel 22.

Through the processing as described above, a jump display operation is performed in the ultrasound diagnosis apparatus 100 so that a state in which an ultrasound image based on one representative frame is displayed changes to a state in which an ultrasound image based on another representative frame is displayed. This makes it easy to perform the operation and processing of designating, from among a plurality of frames stored in the cine memory 34, a frame that includes data indicating a lesion candidate region, and displaying an ultrasound image based on the designated frame.

### (5) Background Processing

The foregoing description has described the operation in which the frame analyzer module 42 performs the grouping processing, the representative frame selection processing, and the lesion measurement processing when the operation mode of the ultrasound diagnosis apparatus 100 is the freeze mode. The frame analyzer module 42 may perform background processing in which the grouping processing, the representative frame selection processing, and the lesion measurement processing are performed when the operation mode of the ultrasound diagnosis apparatus 100 is the real-time measurement mode. In the following description, the background processing will be described mainly with reference to FIGS. 1 and 4.

Each time a frame is newly stored in the cine memory 34, the frame analyzer module 42 performs the grouping processing, the representative frame selection processing, and the lesion measurement processing on frames stored in the cine memory 34. In this embodiment, when the number of frames stored in the cine memory 34 is less than the maximum count N, the above-described processing is performed on as many frames as stored in the cine memory 34. As a result, each time a frame is newly generated and is newly stored in the cine memory 34, the group-of-interest table, the representative frame table, and the measurement information table are updated.

When the ultrasound diagnosis apparatus 100 operates in the real-time measurement mode, the user linearly moves the ultrasound probe 10 on the subject 90 at a constant speed. When a region where a lesion is suspected is identified in ultrasound images displayed on the display 16, the user operates the operation panel 22 to switch the operation mode of the ultrasound diagnosis apparatus 100 from the real-time measurement mode to the freeze mode. The ultrasound diagnosis apparatus 100 whose operation mode is switched to the freeze mode causes the display 16 to display an ultrasound image based on a representative frame that is designated in response to the user's operation on the operation panel 22.

As described above, the processor 24 performs display processing of storing a plurality of ultrasound frames in the cine memory 34 and sequentially displaying ultrasound images based on the plurality of frames on the display 16 with the passage of time. In addition to the display processing, the processor 24 concurrently performs the grouping processing and the representative frame selection processing on the plurality of frames stored in the cine memory 34. In addition to the display processing, the processor 24 concurrently performs the lesion measurement processing (measurement processing) on a lesion candidate region (specific region) based on the representative frame selected by the representative frame selection processing.

Through the processing as described above, ultrasound images based on frames sequentially generated by the frame generation module 30 are sequentially displayed on the display 16, and the group-of-interest table, the representative frame table, and the measurement information table are updated. As such, the grouping processing, the representative frame selection processing, and the lesion measurement processing do not have to be performed anew when the operation mode is switched from the real-time measurement mode to the freeze mode. Therefore, after the operation mode is switched to the freeze mode, the processing of displaying an ultrasound image based on a representative frame on the display 16 is performed quickly. Further, the processing of displaying, in addition to the ultrasound image based on the representative frame, lesion measurement information on the display 16 is performed quickly.

### (6) Second Embodiment

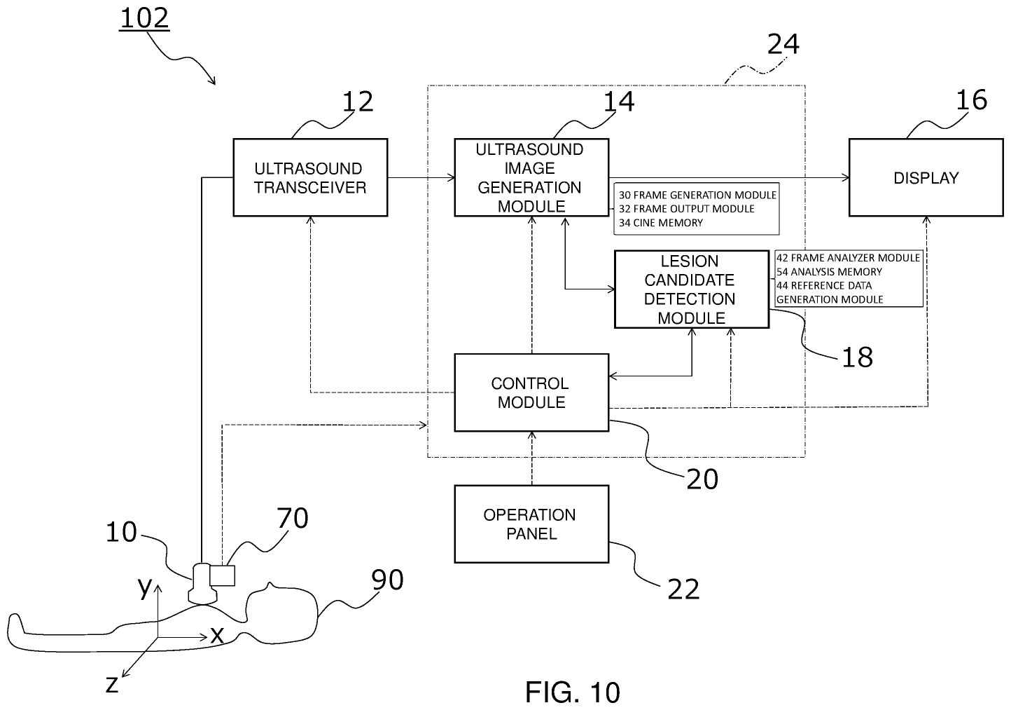

FIG. 10 illustrates a structure of an ultrasound diagnosis apparatus 102 including a position sensor 70 attached to the ultrasound probe 10. The position sensor 70 detects a z axis coordinate value of the ultrasound probe 10 and outputs it to the processor 24. In this embodiment, the z axis is a coordinate axis (spatial axis) that extends in a direction perpendicular to the x-y plane. The frame generation module 30 generates a frame and stores in the cine memory 34, in association with each other, the generated frame and a z axis coordinate value that is obtained by the position sensor 70 at the time when the frame is generated.

When the frames stored in the cine memory 34 include either a frame having a z axis coordinate value that is identical to that of the latest frame or a frame having a z axis coordinate value that is different from that of the latest frame by a difference that falls within a predetermined range, the frame generation module 30 deletes that frame that has been stored, and stores the latest frame in the cine memory 34. Alternatively, when the frames stored in the cine memory 34 include either a frame having a z axis coordinate value that is identical to that of the latest frame or a frame having a z axis coordinate value that is different from that of the latest frame by a difference that falls within a predetermined range, the frame generation module 30 may keep the frame that has been previously stored in the cine memory 34 in the stored state without storing the latest frame.

As described above, the frame generation module 30 stores, in association with each other in the cine memory 34, a frame and a z axis coordinate value of the ultrasound probe 10 (the position of the ultrasound probe 10) that is obtained at the time when the frame is generated. When a z axis coordinate value that is associated with a newly generated frame is identical to a z axis coordinate value that is associated with a frame that has been previously stored in the cine memory 34, or when those z axis coordinate values are different from each other by a difference that falls within a predetermined range, the frame generation module 30 performs storing processing of storing, in the cine memory 34, only one of the newly generated frame and the frame that has been previously stored in the cine memory 34.

While, in the first embodiment, the grouping processing, the representative frame selection processing, and the lesion measurement processing are performed on a plurality of frames arranged on the temporal axis, processing that is similar to that performed on a plurality of frames arranged on the temporal axis may be performed on a plurality of frames arranged on the z axis as in the second embodiment as well. In the grouping processing, the representative frame selection processing, and the lesion measurement processing, the z axis and the temporal axis serving as spatio-temporal axes are treated as numerical axes that are unrelated to the concept of time or space. As such, the grouping processing, the representative frame selection processing, and the lesion measurement processing that are similar to those performed in the first embodiment in which a plurality of frames are arranged on the temporal axis may be performed in the second embodiment in which a plurality of frames are arranged on the z axis.

In the second embodiment, the frame generation module 30 avoids repeated storing of frames whose z axis coordinate values are identical or close to each other in the cine memory 34, and performs no wasteful data storage in the cine memory 34.

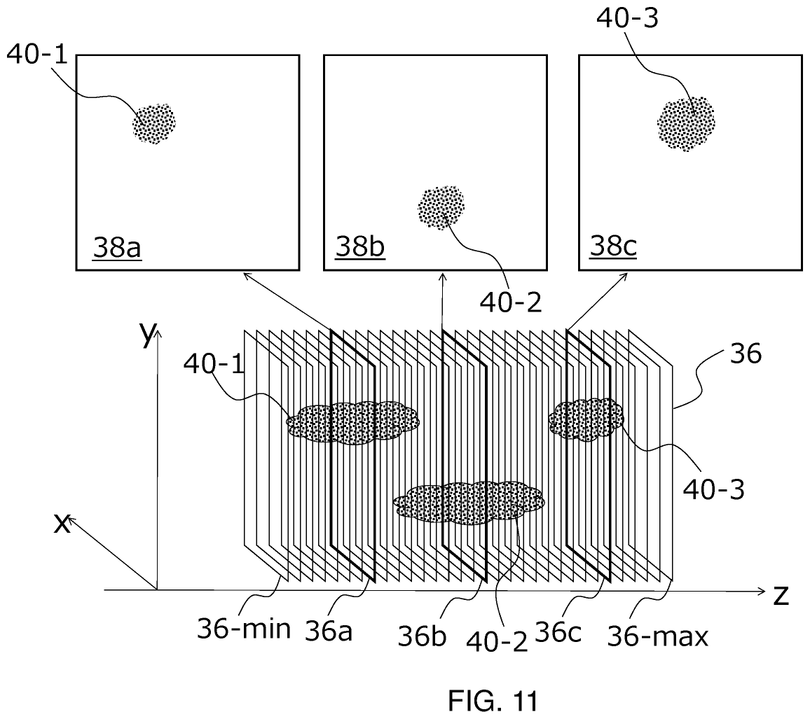

The lower half of FIG. 11 conceptually illustrates two-dimensional ultrasound images of frames 36 stored in the cine memory 34. The horizontal axis is the z axis, and the x-y plane is defined as vertical to the z axis. Ultrasound images represented by individual frames 36 extend in parallel with the x-y plane, and a plurality of frames are successive along the z axis. A frame 36-min that is located leftmost has a minimum z axis coordinate value, and a frame 36-max that is located rightmost has a maximum z axis coordinate value. The upper half of FIG. 11 conceptually illustrates ultrasound images 38a, 38b, and 38c represented by representative frames 36a, 36b, and 36c.

If the representative frame 36a is designated in response to an operation of the operation panel 22, the display 16 displays the ultrasound image 38a. Similarly, if the representative frame 36b or 36c is designated, the display 16 displays the ultrasound image 38b or 38c.

The lower half of FIG. 11 illustrates lesion candidate regions 40-1, 40-2, and 40-3 for individual frames. The lesion candidate region 40-1 appears in the ultrasound image 38a. The lesion candidate region 40-2 appears in the ultrasound image 38b. The lesion candidate region 40-3 appears in the ultrasound image 38c. As described above, one of the representative frames stored in the cine memory 34 is designated by the user, and an ultrasound image is displayed in accordance with the designated representative frame to thereby diagnose a lesion candidate region.

### (7) Display of Doppler Images, Elastic Images, or Other Images

While, in the above-described embodiments, the frame generation module 30 generates frames representing ultrasound images of the tomographic plane of the subject 90, the frame generation module 30 may generate frames representing other images such as Doppler images or elastic images. A Doppler image overlays on a tomographic image of the subject 90, for example, an arrow or coloring indicating how blood flows. An elastic image overlays on a tomographic image of the subject 90, for example, coloring indicating tissue hardness. To display Doppler images or elastic images, the ultrasound transceiver 12 outputs to the ultrasound probe 10 transmission signals for generating frames representing Doppler images or elastic images and obtains reception signals from the ultrasound probe 10 in accordance with the transmission signals. The ultrasound transceiver 12 further generates signals for generating frames representing Doppler images or elastic images and outputs the generated signals to the frame generation module 30.

1. An ultrasound diagnosis apparatus comprising a processor for performing, on a plurality of ultrasound frames that are sequentially generated by transmission and reception of ultrasound:
grouping processing of identifying a group of interest that is composed of a plurality of frames that satisfy at least one predetermined condition of interest; and
representative frame selection processing of selecting a representative frame from among the plurality of frames that constitute the group of interest,
wherein the at least one predetermined condition of interest comprises a condition that each of the plurality of frames includes data indicating a specific region identified by region recognition processing in which a characteristic region in an image is recognized as the specific region, and
wherein the representative frame selection processing comprises processing that selects the representative frame based on geometrical properties of the specific regions.

2. The ultrasound diagnosis apparatus according to claim 1, wherein the grouping processing comprises processing that identifies, from among the plurality of ultrasound frames, frames that constitute the group of interest based on a positional relationship of the specific regions for the ultrasound frames that are adjacent to each other on a temporal axis or a spatial axis.

3. The ultrasound diagnosis apparatus according to claim 1, wherein the grouping processing comprises:
processing of generating a detection information table listing information representing geometrical properties of the specific regions indicated by data included in the ultrasound frames in association with frame identification information that identifies the ultrasound frames; and
processing of identifying the group of interest from among the plurality of ultrasound frames based on the detection information table.

4. The ultrasound diagnosis apparatus according to claim 1, further comprising:
a memory for storing the plurality of ultrasound frames,
wherein the processor reads the representative frame from the memory based on information that identifies the representative frame.

5. The ultrasound diagnosis apparatus according to claim 4, further comprising:
a display for displaying images based on the ultrasound frames,
wherein the processor performs display processing of storing the plurality of ultrasound frames in the memory and sequentially displaying on the display images based on the plurality of ultrasound frames with the passage of time, and
wherein, in addition to the display processing, the processor concurrently performs the grouping processing and the representative frame selection processing on the plurality of ultrasound frames stored in the memory.

6. The ultrasound diagnosis apparatus according to claim 5,
wherein, in addition to the display processing, the processor concurrently performs measurement processing on one or more of the specific regions based on the representative frame selected by the representative frame selection processing.

7. The ultrasound diagnosis apparatus according to claim 4, further comprising:
a display for displaying images based on the ultrasound frames,
wherein the processor performs display processing of storing the plurality of ultrasound frames in the memory and sequentially displaying images based on the plurality of ultrasound frames on the display with the passage of time,
wherein the processor performs freeze processing of stopping the display processing based on an operation of a user and keeping a state in which either an image that was displayed on the display when the operation has occurred or an image based on a previously generated ultrasound frame is displayed on the display; and
wherein, after the operation has occurred, the grouping processing and the representative frame selection processing are performed on the plurality of ultrasound frames stored in the memory.

8. The ultrasound diagnosis apparatus according to claim 4, further comprising:
an ultrasound probe for transmitting and receiving ultrasound to and from a subject; and
a position sensor for detecting a position of the ultrasound probe,
wherein the processor stores, in association with each other in the memory, an ultrasound frame and a position of the ultrasound probe detected when the ultrasound frame is generated, and
wherein, when a position of the ultrasound probe corresponding to a newly generated ultrasound frame is identical to a position of the ultrasound probe corresponding to an ultrasound frame that has been previously stored in the memory, or when a difference between those positions of the ultrasound probe falls within a predetermined range, the processor stores, in the memory, only one of the newly generated ultrasound frame and the ultrasound frame that has been previously stored in the memory.

9. An ultrasound diagnosis program that causes a processor to perform, on a plurality of ultrasound frames that are sequentially generated by transmission and reception of ultrasound:
grouping processing of identifying a group of interest that is composed of a plurality of frames that satisfy at least one predetermined condition of interest; and
representative frame selection processing of selecting a representative frame from among the plurality of frames that constitute the group of interest,
wherein the at least one predetermined condition of interest comprises a condition that each of the plurality of frames includes data indicating a specific region identified by region recognition processing in which a characteristic region in an image is recognized as the specific region, and
wherein the representative frame selection processing comprises processing that selects the representative frame based on geometrical properties of the specific regions.
